(19) United States
(12) Patent Application Publication
(10) Pub. No.: US 2022/0287761 A1
(43) Pub. Date: Sep. 15, 2022

(54) DISTAL WIRE ROUTING FOR STRAIGHT JAW FORCEPS

(71) Applicant: GYRUS ACMI, INC,D/B/A OLYMPUS SURGICAL TECHNOLOGIES AMERICA, Westborough, MA (US)

(72) Inventors: Theodore C. Blus, Arden Hills, MN (US); Daniel C. weber, Champlin, MN (US)

(21) Appl. No.: 17/653,486

(22) Filed: Mar. 4, 2022

Related U.S. Application Data
(60) Provisional application No. 63/200,470, filed on Mar. 9, 2021.

(51) Int. Cl. A61B 18/14; A61B 18/12

(52) U.S. Cl. CPC A61B 18/1445; A61B 18/1206; A61B 2018/00178

(57) ABSTRACT

The present disclosure includes, among other things, a surgical forceps device and system. The device can include first and second opposing jaws with correlating jaw flanges meeting at a pivot point, and a distal wire guide situated within the device proximal of the jaw flanges. Wiring can run on an outside surface of the jaws, through the distal wire guide, and be routed internally as the wiring extends proximally down the device. The wire guide can act as a spacer to help guide the wiring in the device.

### CROSS-REFERENCE TO RELATED APPLICATION

This application claims the benefit of priority to U.S. Provisional Patent Application Ser. No. 63/200,470, filed Mar. 9, 2021, the contents of which are hereby incorporated by reference in their entirety.

### BACKGROUND

The present invention relates generally to systems and methods for surgical medical devices and specifically to surgical forceps having an actuatable jaw and/or blade.

Medical devices for diagnosis and treatment, such as surgical forceps, can be used for medical procedures such as laparoscopic and open surgeries. Such forceps can be used to manipulate, engage, grasp, or otherwise affect an anatomical feature, such as a vessel or other tissue. A surgical forceps can further include an end effector such as rotatable, openable, closeable, extendable, retractable, or other components. In some cases, surgical forceps can be capable of supplying an input such as ultrasound or electromagnetic energy.

Surgical forceps can include a jaw on a distal end of the forceps. Such a jaw can be articulated or actuated through element on or in a handpiece of the forceps, and can be manipulated to open and close, engaging vessels or other tissue. In some cases, the jaw can be rotationally actuatable. In some cases, the jaw can include an extendable or retractable blade, such that the blade can extend distally between the jaw.

Surgical forceps often include electrical connections between the jaw and the handpiece, such as through electrical wiring. Depending on the specific functions of the forceps, wiring can take up space within the design of the forceps.

### SUMMARY OF THE DISCLOSURE

The present disclosure provides surgical forceps and associated methods, where the wire routing in the surgical jaw extends from outside the jaw to inside the device from distal to proximal portions. The wiring can be secured through a distal wire guide. Skiving of wire insulation or the wire itself can be inhibited or reduced using angled cutting in the distal end of the outer shaft of the device. In some cases, one of the jaws can be welded to allow for single action forceps, in this case, the jaw can be recentered with shifting the jaw away from the weld side, or bumping in the outer shaft.

In surgical forceps, wiring can be used to electrically connect one or more electrodes on the jaw of the forceps. The wiring can take up space within the forceps, and can be routed along the length of the forceps from the handle down to the electrodes. In some cases, the wiring is routed inside the forceps, and in some cases, the wiring is routed outside the forceps. In some cases, wiring is routed from inside the handle to outside the jaws of the forceps, depending on the availability of space within the forceps. Some types of wire routing can allow for easily disrupted wiring, or wire skiving along the length of the forceps.

The discussed wire routing arrangements herein can reduce wire skiving within the device. In some cases, the proposed surgical forceps and associated methods can allow for more efficient and protected wire routing with the forceps. Additionally, the proposed surgical forceps can allow for centered jaws for single action electrosurgery.

In an example, a forceps system can include a forceps, including first jaw and an opposing second jaw, at least one of the first and second jaw actuatable for closing the forceps and including first and second jaw flanges extending proximally from a pivot point; an elongated body with a distal end and a proximal end, wherein the forceps extend from the distal end of the elongated body; a wire guide situated within the distal end of the elongated body; and wire extending along a lateral outside surface of at least one of the first and second flanges, through the wire guide, and along an inside surface of the body towards the proximal end, wherein the wire guide includes a spacer separating the wire from the inside surface of the body.

In an example, a forceps device can include a forceps including first jaw and an opposing second jaw, wherein the first jaw is welded to a frame and the second jaw is actuatable for opening and closing the forceps; an elongated body extending between a distal portion and a proximal portion, wherein the forceps extend from the distal portion, the body comprising the frame; and a re-centering feature in the device to offset the location of the weld and center the opening of the jaw in the device.

In an example, a method of electrically connecting a forceps device can include connecting one or more wires to an end effector on a jaw of the forceps; running the one or more wires proximally along an outside surface of the jaw; guiding the one or more wires from the outside surface of the jaw to a distal wire guide comprising a spacer and threading the one or more wires through the distal wire guide; running the one or more wires from the distal wire guide proximally to an inside surface of an elongated body of the forceps; and connecting the one or more wires to a power source.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a side view of a surgical forceps showing jaws in an open position in an example.

FIG. 2A-2B illustrate views of a surgical forceps with a straight jaw in an example.

FIGS. 3A-3D illustrate perspective views of wiring in a surgical forceps with a straight jaw in an example.

FIGS. 4A-4C illustrate views of a distal wire guide for a surgical forceps.

FIGS. 5A-5B illustrate views of a distal end cut for wire routing in a surgical forceps in an example.

FIGS. 6A-6B illustrate views of a distal end cut for wire routing in a surgical forceps in an example.

FIGS. 7A-7D illustrate schematic drawings of a single action surgical forceps with a welded jaw in an example.

### DETAILED DESCRIPTION

The present disclosure describes, among other things, systems and methods including a surgical forceps with wire routing running from outside the jaw of the surgical forceps into the handpiece of the surgical forceps. Provided are additional methods and system of wire routing within a surgical forceps for more effective surgical forceps.

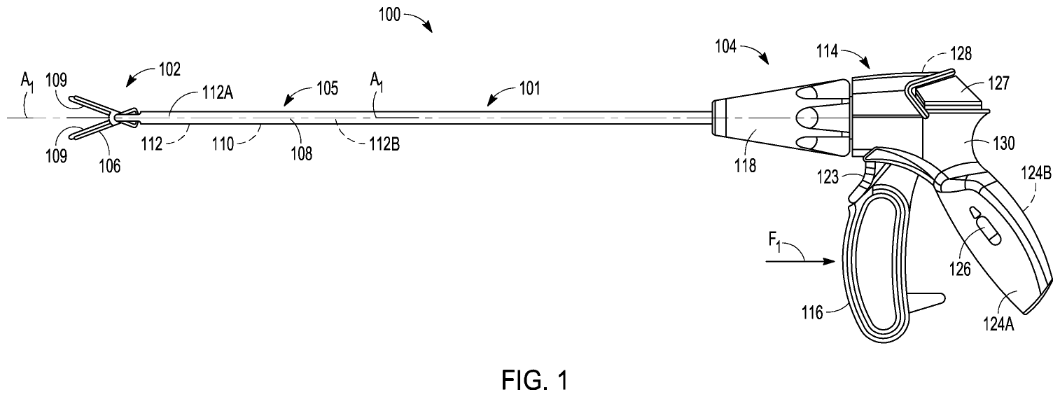

FIG. 1 illustrates a side view of a forceps 100 showing jaws in an open position. The forceps 100 can include an end effector 102, a handpiece 104, and an intermediate portion 105. The end effector 102 can include jaws 106 (including grip plates 109a, 109b), an outer shaft 108, an inner shaft 110, and a blade assembly 112. The handpiece 104 can include a housing 114, a lever 116, a rotational actuator 118, a trigger 123, an activation button 127, a welded handle 124a and 124b, and a handle locking mechanism 126. The housing 114 can include a first housing portion 128, and a second housing portion 130. FIG. 1 also shows orientation indicators Proximal and Distal and a longitudinal axis A1.

The surgical forceps 100 can be single acting or dual acting. A single acting surgical forceps has one welded jaw and one mobile jaw opposite each other, while a dual acting surgical forceps has two mobile jaws opposite each other.

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated along the longitudinal axis A1 of the forceps 100. Also, the handpiece can operate the jaws 106, such as by moving the jaws 106 between open and closed position. The handpiece 104 can also be used to operate the blade assembly 112 for cutting tissue and can operate the electrode on the jaw 106 for applying electromagnetic energy to tissue. The end effector 102, or a portion of the end effector 102, can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not be housed within the housing 114.

The drive shaft 110 can extend through the housing 114 and out of a distal end of the housing 114, or distally beyond housing 114. The jaws 106 can be connected to a distal end of the drive shaft 110. The outer shaft 108 can be a hollow tube positioned around the drive shaft 110. A distal end of the outer shaft 108 can be located adjacent the jaws 106. The distal ends of the drive shaft 110 and the outer shaft 108 can be rotationally locked to the jaws 106. The rotational actuator 118 can be positioned around the distal end of the housing 114. The outer shaft 108 can extend distally beyond the rotational actuator 118. The blade shaft 112b can extend through the drive shaft 110 and the outer shaft 108. A distal end of the blade shaft 112b can be located near the jaws 106. A proximal end of the blade shaft 112b can be within housing 114.

In operation of the end effector 102, a user can displace the lever 116 proximally by applying a Force F1 to the lever 116 to actuate the drive shaft 110 to drive the jaws 106 from the open position to the closed position, which can allow the user to clamp down on and compress a tissue. The handpiece 104 can also allow a user to rotate the rotational actuator 118 to cause the end effector 102 to rotate, such as by rotating both the inner (drive) shaft 110 and the outer shaft 108 together.

In some examples, with the tissue compressed, a user can depress the activation button 127 to cause an electromagnetic energy, or in some examples, ultrasound, to be delivered to the end effector 102, such as to the electrode on the jaw 106 and to the tissue. Application of such energy can be used to seal or otherwise affect the tissue being clamped. In some examples, the electromagnetic energy can cause tissue to be coagulated, sealed, ablated, desiccated or can cause controlled necrosis. When desired, the trigger 123 can be moved to translate the blade assembly 112 distally such that the blade 112a can extend between the jaws 106 in order to cut the tissue within the jaws 106. Such a process can be repeated, as desired.

In some examples, the forceps 100, or other medical device, may not include all the features described or may include additional features and functions, and the operations may be performed in any order. The handpiece 104 can be used with a variety of other end effectors to perform other methods.

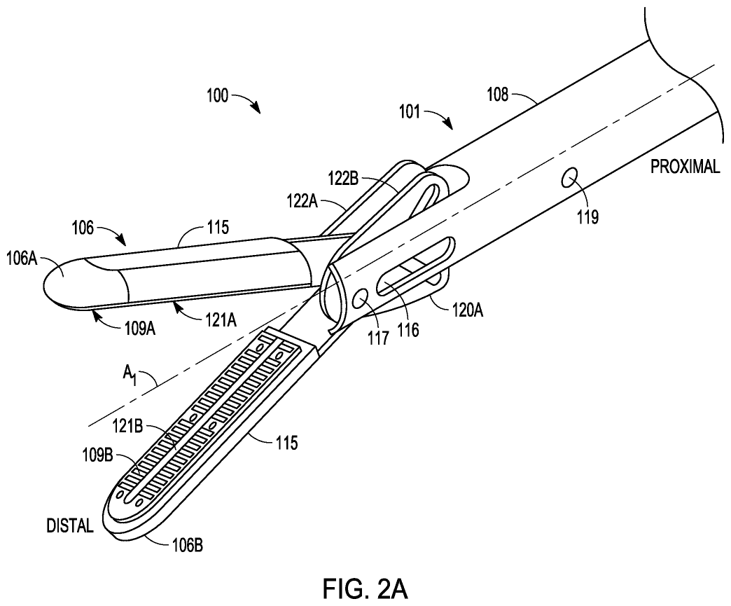

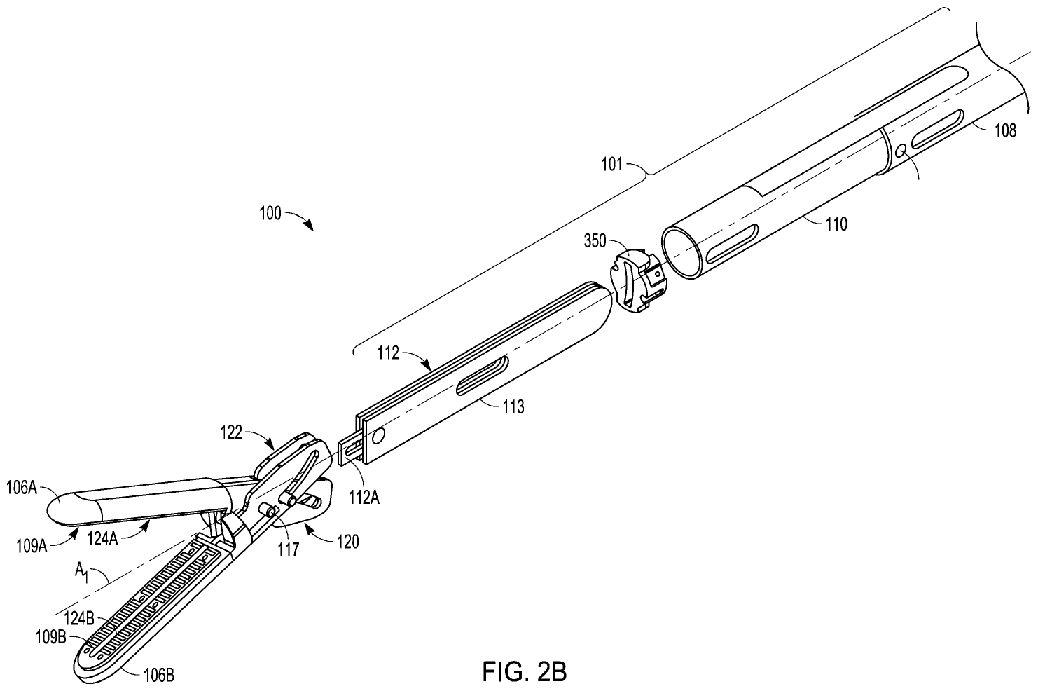

FIG. 2A illustrates a perspective view of a portion of the forceps 100 in an open position, and a closer view of the end effector 102. FIG. 2B illustrates an exploded view of a portion of the forceps 100 in an open position, and a closer view of the end effector 102. FIGS. 2A-2B are discussed below concurrently.

The forceps 100 can include first jaw 106a, second jaw 106b opposite the first jaw 106a, where at least one of the first jaw 106a and the second jaw 106b can be actuatable for closing the forceps 100. The first jaw 106a can include jaw flanges 120a, 120b, and the second jaw can include jaw flanges 122a, 122b. The jaw flanges 120a, 120b, 122a, 122b, can extend from the pivot point 117. The forceps can include an elongated body 101 including the outer shaft 108 and the inner shaft 110 extending between the jaw 106 and a handle (such as handpiece 104). The jaw 106 can extend distally from the elongated body 101. A distal wire guide 150 can be situated within the distal end of the elongated body 101, such as between the jaw flanges 120a, 120b, 122a, 122b, and the inner shaft 110. Wiring 115 can extend laterally outside the surface of the first jaw 106a, and the second jaw 106b, and down at least one of the first and second flanges 120, 122. The wiring 115 can run through the distal wire guide 150 and along an inside surface of the elongated body 101 towards the proximal end of the forceps 100. The wire guide 150 can include a spacer 151 for separating the wiring 115 from the inside surface of the elongated body 101. The wire guide 150 with spacer 151 can, for example, help guide the wiring 115 around the jaw flanges 120a, 120b, 122a, 122b as the wiring 115 is routed from the outside of the forceps 100 to the inside of the elongated body 101. The wire guide 150 can, for example, help move the wiring 115 apart or separate from the central longitudinal axis A1. Examples of such a wire guide are discussed in more depth below.

The forceps 100 can include the end effector 102, that can be connected to a handle (such as the handpiece 104). The end effector 102 can include first jaw 106a and second jaw 106b, an outer shaft 108, grip plates 109a and 109b, an inner shaft 110, a blade assembly 112, a drive bar 113, a pivot point 117, a drive pin 119, and a rotational actuator 118. The first jaw 106a can include jaw flanges 10120a and 10120b, and the jaw 106b can include jaw flanges 122a and 122b. The wiring 115 can run around the jaw flanges 10120a, 10120b, 122a, 122b, within the elongated body 101. The grip plate 109a can include a blade slot 121a and the grip plate 109b can include a blade slot 121b. The blade assembly 112 can include a blade 112a. FIGS. 2A-2B also show orientation indicators Proximal and Distal and a central longitudinal axis A1.

The components of the forceps 100 can each be comprised of materials such as one or more of metals, plastics, foams, elastomers, ceramics, composites, combinations thereof, or the like.

The jaws 106a and 106b can be rigid or semi-rigid members configured to engage tissue. The jaws 106a and 106b can be coupled to the outer shaft 108, such as pivotably coupled, via the pivot point 117. The pivot point 117 can extend through a portion of the jaws 106a and 106b (such as a bore of each of the jaws 106a and 106b) such that the pivot point 117 can be received by outer arms of the outer shaft 108. In other examples, the jaws 106a and 106b can be pivotably coupled to the outer shaft 108 via a boss or bosses of the outer shaft 108. In another example, the jaws 106a and 106b can include a boss (or bosses) receivable in bores of the outer shaft 108 to pivotably couple the jaws 106a and 106b to the outer shaft 108. In another example, outer shaft 108 can include a boss (or bosses) receivable in bores of the jaws 106a and 106b to pivotably couple the jaws 106a and 106b to the outer shaft 108.

The jaw flanges 120a and 120b (which can be a set of jaw flanges, e.g., two jaw flanges) can be rigid or semi-rigid members located at a proximal portion of the first jaw 106a. Similarly, the jaw flanges 122a and 122b can be rigid or semi-rigid members located at a proximal portion of the second jaw 106b. In some examples, the jaw flanges 120, 122 can be positioned laterally outward of the inner jaw flanges 122. In other examples, the jaw flanges 120 and 122 can be interlaced. In some cases, the jaw flanges 120, 122, can be nested within each other, or alternating.

The grip plates 109a and 109b of the first and second jaws 106a and 106b can each be a rigid or semi-rigid member configured to engage tissue and/or the opposing jaw to grasp tissue, such as during an electrosurgical procedure. One or more of the grip plates 109a and 109b can include one or more of serrations, projections, ridges, or the like configured to increase engagement pressure and friction between the grip plates 109a and 109b and tissue. The jaw flanges 120a, 120b, of the upper jaw 106a can extend proximally away from the grip plate 109a and 109b, and in some examples, substantially downward when the upper jaw 106a is in the open position. Similarly, the jaw flanges 122 of the lower jaw 106b can extend proximally away from the grip plate, and in some examples, substantially upward when the upper jaw 106a is in the open and partially open positions, such that the first and second jaws 106a and 106b and jaw flanges 120 and 122 operate to open and close in a scissoring manner. The first and second jaws 106a and 106b can each include an electrode configured to deliver electricity to tissue (optionally through the grip plates 109a and 109b), and a frame supporting the electrode. The blade slots 121a and 121b of the grip plates 109a and 109b can together be configured to receive a blade between the jaws 106a and 106b when the jaws are moved out of the open position. In some examples, only one blade slot may be used.

The elongated body 101 can extend between the handpiece 104 and the jaw 106. The elongated body 101 can include the outer shaft 108 and the inner shaft 110. The inner shaft can at least partially be situated within the outer shaft 108. The wiring 115 can extend from the wire guide 150 into the inner shaft 110. Each of the inner shaft 110 and the outer shaft 108 can be a rigid or semi-rigid and elongated body having a geometric shape of a cylinder, where the shape of the inner shaft 110 matches the shape of the outer shaft 108. In some examples, the inner shaft 110 and the outer shaft 108 can have other shapes such as an oval prism, a rectangular prism, a hexagonal prism, an octagonal prism, or the like. In some examples, the shape of the inner shaft 110 can be different from the shape of the outer shaft 108.

The inner shaft 110 can extend substantially proximally to distally along the axis A1, which can be a central longitudinal axis. In some examples, the axis A1 can be a central longitudinal axis. Similarly, the outer shaft 108 can extend substantially proximally to distally along the axis A1. In some examples, the axis A1 can be a central axis of one or more of the inner shaft 110 and the outer shaft 108. The inner shaft 110 can include an axial bore extending along the axis A1. The outer shaft 108 can also include an axial bore extending along the axis A1. The inner shaft 110 can have an outer dimension (such as an outer diameter) smaller than an inner diameter of the outer shaft 108 such that the inner shaft 110 can be positioned within the outer shaft 108 and such that the inner shaft 110 can be translatable in the outer shaft 108 along the axis A1. The inner shaft 110 can also be referred to as a drive shaft 110, a cam shaft 110, or an inner shaft 110.

The blade 112a can be an elongated cutting member at a distal portion of the blade assembly 112. The blade 112a can include one or more sharpened edges configured to cut or resect tissue or other items. The blade assembly 112 can be located within the outer shaft 108 (and can be located within the inner shaft 110). The blade 112a can extend along (and optionally parallel with) the axis A1. The blade 112a can be translatable with respect to the inner shaft 110 and the outer shaft 108 to extend between (or into) the first jaw 106a and the second jaw 106b, such as along the blade slots 121a and 121b. In some examples, the blade 112a can extend axially through the inner shaft 110 offset from the axis A1. In some examples, the blade 112a the blade can extend axially through the jaw flanges 120 and 122 to extend into the jaws 106, such that the blade 112a is in a position laterally inward of the first set of jaw flanges 120 and the second set of jaw flanges 122. In other examples, the blade 112a can be positioned laterally inward of some jaw flanges (120 or 122) and laterally outward of other jaw flanges (120 or 122). In some examples, each jaw 106 can include only a single jaw flange 120 and 122. In such an example, the blade 112a can extend between (laterally inward of) the two jaw flanges 120 and 122 or can extend laterally outward of the jaw flanges 120 and 122.

The blade 112a can also be a translating member or electrosurgical component other than a blade. For example, the blade 112a can be an advancing electrosurgical electrode configured to cut tissue, such as a blunt electrode, an electrosurgical blade, a needle electrode, or a snare electrode.

In operation, the inner shaft 110 can be translated using an actuator (such as the lever 116 of FIG. 1). The inner shaft 110 can translate with respect to the outer shaft 108 to move the drive pin 119. The drive pin 119 can engage the jaw flanges 120 and 122 to move the jaw flanges 120 and 122 between open and closed positions, which can cause the jaws 106a and 106b to move between open and closed positions.

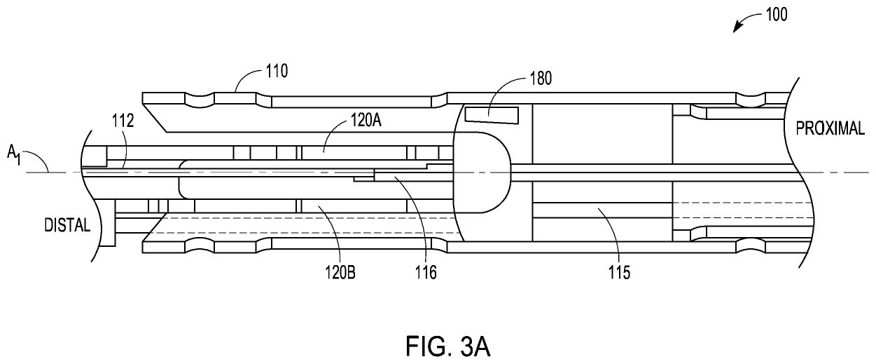

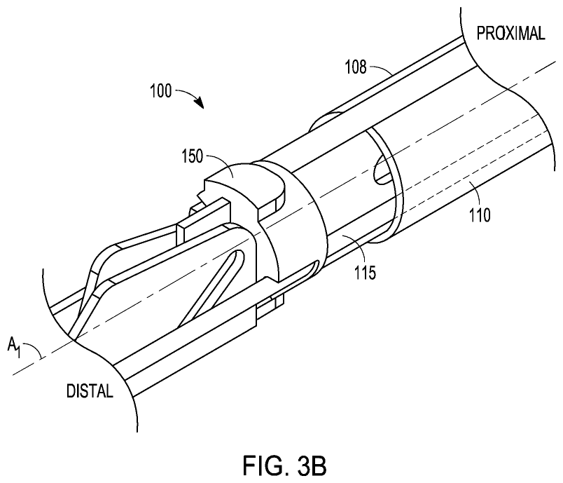

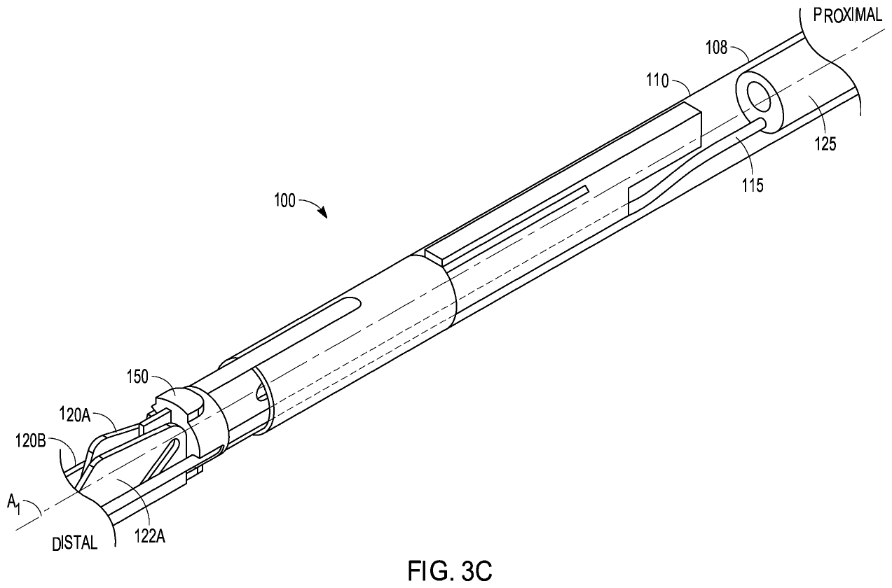

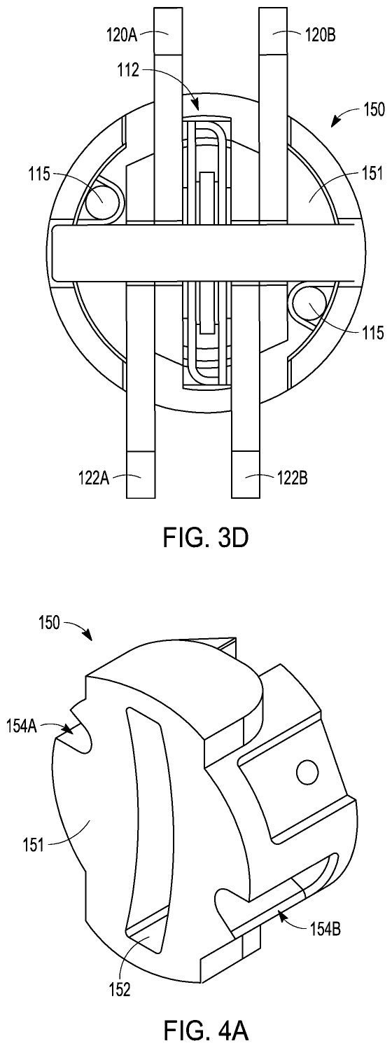

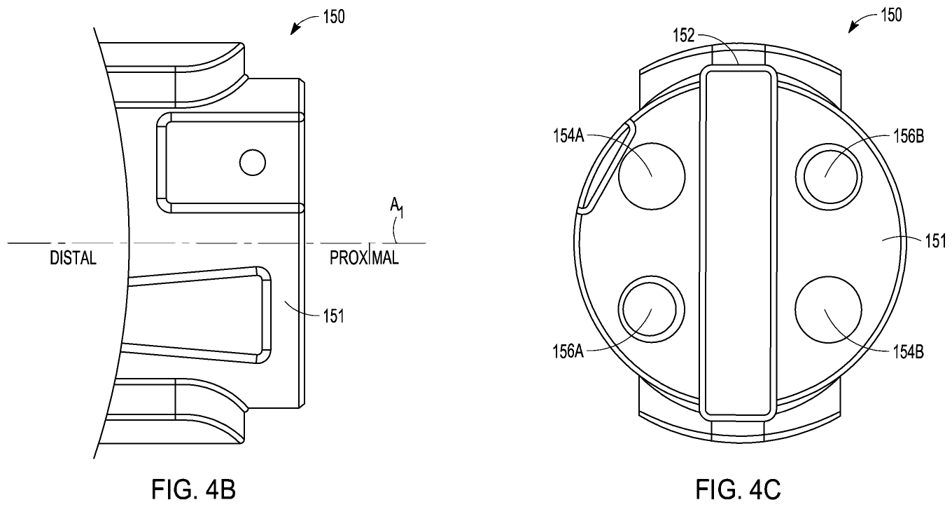

A distal wire guide can be situated within the forceps 100 proximal the jaw 106 and the jaw flanges 120, 122. The distal wire guide can be situated within the inner shaft 110 and the outer shaft 108, to guide wires from the jaw 106 into the elongated body defined by the inner and outer shafts 108, 110, towards the handpiece 104. Example distal wire guides are discussed below with reference to FIGS. 3A-4C.

FIGS. 3A-3D illustrate perspective views of wiring 115 in the surgical forceps 100 with a straight jaw, while FIGS. 4A-4C illustrate views of a distal wire guide 150 for the surgical forceps 100. The surgical forceps 100 can include advantageous wire routing that is guided by the distal wire guide 150.

FIG. 3A depicts a side cut-away view of a portion of the surgical forceps 100, the portion near the jaw flanges 120, 122 and the outer shaft 108. FIG. 3B depicts a perspective cut-away view of a portion of the surgical forceps 100 near the jaw flanges 120, 122, and the outer shaft 108. FIG. 3C depicts a depicts a perspective cut-away view of a portion of the surgical forceps 100 extending from the jaw flanges 10120, 122, proximally down the elongated shaft of the outer shaft 108. FIG. 3D depicts a schematic view down the shaft of the forceps 100 at the wire guide 150. FIG. 4A depicts a perspective view of a wire guide 150. FIG. 4B depicts a side view of a wire guide 150. FIG. 4C depicts a front face view of the wire guide 150. FIGS. 3A-4C will be discussed concurrently below.

The forceps 100 can include the outer shaft 108, inner shaft 110, jaw flanges 120a, 120b, blade assembly 112, in addition to the wiring 115, the connector 125, and the distal wire guide 150. The distal wire guide 150 can include drive bar slot 152, wire holders 154a, 154b, and pins 456a, 456b. FIGS. 3A-4C also show orientation indicators Proximal and Distal and a longitudinal axis A₁.

In surgical forceps such as forceps 100 discussed above, the forceps can often have a connector 125 where wiring is connected to allow electrical current to flow to electrodes or other components on the end effector 102, and to power the forceps 100. The wiring 115 can be threaded along the length of the forceps 100 from the jaw 106 to the handpiece 104 to allow for electrical connection.

The surgical forceps 100 can be a straight-jaw type forceps. The surgical forceps 100 can be a single acting or dual acting forceps; for example, one or both of the jaws 106a, 106b, can be actuatable during operation. Straight-jaw type forceps can have limited space inside the forceps 100 between the jaw 106 and the handpiece 104. Between the moveable jaw 106 and the elongated portions of the outer shaft 108 and inner shaft 110, a variety of moving parts are contained within the forceps 100, including, for example, the blade assembly 112, the drive bar 113, the pivot point 117, and the jaw flanges 10120, 122. Each of these components moves during operation as described above with reference to FIGS. 1-2B. Thus, if the wiring 115 is inside the forceps 100, the wiring 115 can accidentally or unintentionally interact with the moving parts, which can potentially cut, kink, or otherwise disrupt the wiring 115 providing power to the forceps 100 and the end effector 102.

Moreover, between the jaw 106 and the elongated outer shaft 108, there can be minimal room for wiring inside the forceps 100. For this reason, the wiring 115 can be run on the outside of the two jaws 106a, 106b, of the forceps 100, from the distal end towards the pivot point 117. However, running the wiring 115 on the outside of the forceps 100 the entire length of the device can cause unintentional wire exposure during operation.

The distal wire guide 150 can be inserted into the distal end of the outer shaft 108 of the forceps 100 to direct the wiring 115 in the forceps 100 from the outside of the jaw 106 towards the inside of the inner shaft 110 after the wiring 115 has cleared moving parts such as the jaw flanges 120, 122. As shown in FIGS. 3D and 4A-4C, the wire guide 150 can include wire holders 154a, 154b, such as for directing individual electrode wires, and a slot 152 for the blade assembly 112 inner (drive) shaft 110 and the blade assembly 112 to passthrough. The distal wire guide 150 can allow for separation of the wiring 115 from the moving parts in this region: the reciprocating blade assembly 112, the pivoting jaw flanges 10120, 122, and the inner surface of the outer shaft 108. For example, the distal wire guide 150 can help position the wiring 115 as it extends proximally such that the wiring 115 is moved away from an outside surface of the elongated body relative the central longitudinal axis A1. In this way, the wiring 115 can internally extend towards the handle.

In FIGS. 3A-3C, the wiring 115 can be seen moving proximally along the length of the forceps 100 from the outside of the jaw 106, outside past the jaw flanges 120, 122 and the blade assembly 112. The wiring 115 can be routed from outside to inside just past these moving parts at the distal wire guide 150, into the inner shaft 110, so as to avoid the movement of the outer shaft 108. The wiring 115 can then continue to run along the length of the forceps 100 to the connector 125, where the wiring 115 can be connected to a power source through the handpiece 104. The power source can be, for example, through a ground outlet, or an electrical generator providing power to the wiring 115.

The wiring 115 can include a single wire, multiple wires, a bundle of wires, or combinations thereof. The wiring 115 can include two sides, such as one wiring 115 running along each of the jaws 106a, 106b. In FIG. 3C, the wiring 115 can be seen on either side of the jaw flanges 120, 122, which are depicted in FIG. 3C as alternating, although other configurations of jaw flanges can be used. The distal wire guide 150 can allow for the two portions of the wiring 115 to go in opposite ways above or below the pivot point 117, and the center of the distal wire guide 150. Once the wiring 115 is passed through the distal wire guide 150, it can continue along a central lumen in the inner shaft 110 towards the proximal end of the forceps 100. The wiring 115 can extend to the handpiece 104.

The distal wire guide 150 itself can contain a central slot 152 and two wire holders 154a, 154b. The distal wire guide 150 can have a curved perimeter to allow for clearance with the proximal ends of the pivoting jaw flanges 120, 122. The central slot 152 can allow passage of the drive bar 113 and the blade 112a in the blade assembly 112. The drive bar 113 can allow passage of the blade 112a through its center. An example drive bar mechanism is described in U.S. patent application Ser. No. 16/829,799, which is incorporated herein by reference in its entirety. Opposite the wire holders 154, two or more pins 456a, 456b, can be used to secure the distal wire guide 150 in place within the forceps 100.

The use of the distal wire guide 150 can avoid entanglement of the wiring 115 inside the forceps 100, and help avoid entanglement as the wiring 115 is routed from outside the jaw 106 to inside the inner shaft 110. As the jaws 106 open and close during operation, the jaw flanges 10120, 122, can create a scissor effect. The use of the wire guide 150 can help reduce the wiring 115 from skiving along the jaw flanges 120, 122, or other moving parts of the forceps 100 as the wiring 115 moves inboard.

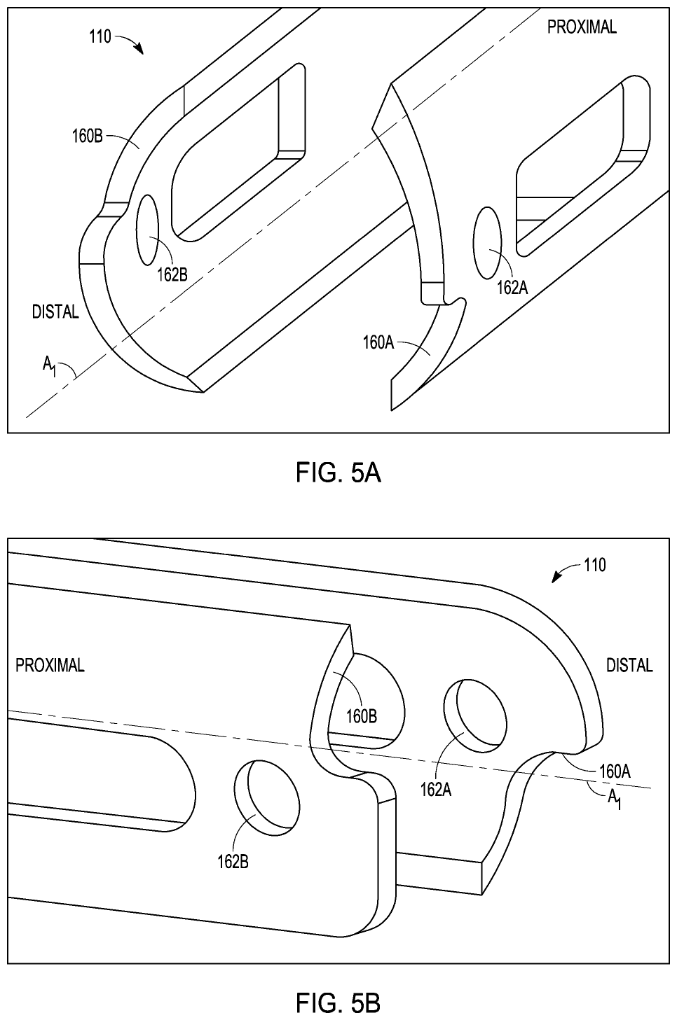

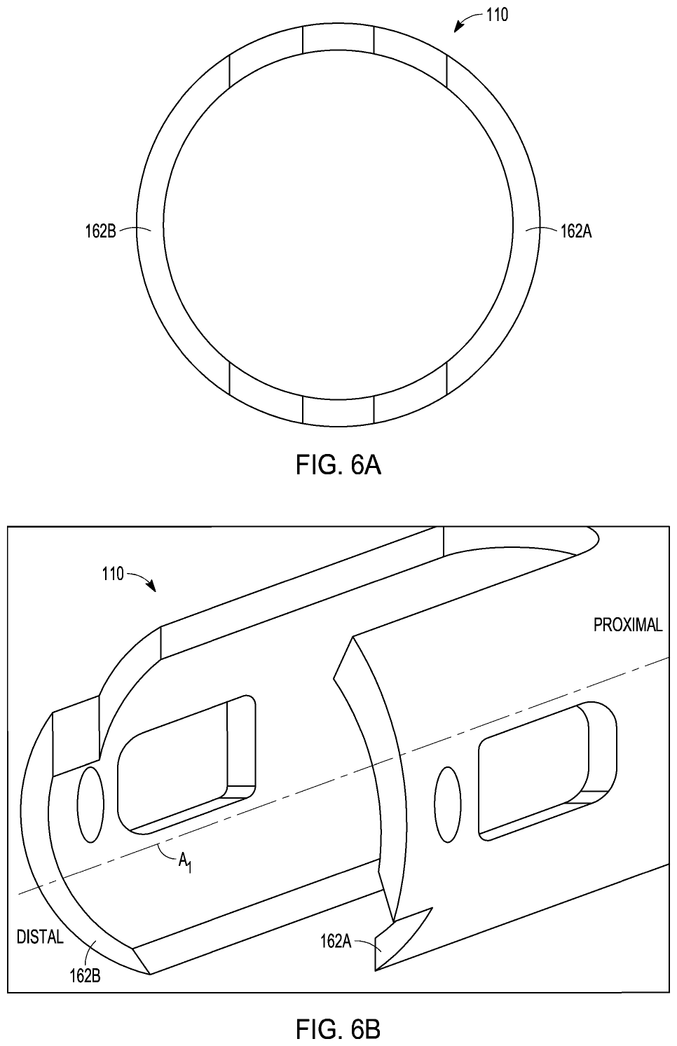

In some cases, the wiring 115 can be subject to skiving at the distal end of the outer shaft 108 as the wiring 115 is routed inward. FIGS. 5A-5B and FIGS. 6A-6B illustrate views of example distal end cutting for wire routing in surgical forceps 100 to help reduce such skiving. In FIGS. 5A-5B, the outer shaft 108 can include cuts 160a, 160b, and holes 162a, 162b. In FIGS. 6A-6B, the outer shaft 108 can include cuts 660a, 660b, and holes 162a, 162b. The cuts 160, 660, are different shapes.

In FIGS. 5A-5B, an additional geometric cut on the distal end of the outer shaft 108 can be made on either side to align with the wire holders 154 of the distal wire guide 150. Here, the example geometric cut can be a curved cut to allow clearance of the wiring 115 as it extends into the inner shaft 110. A curved cut can, for example, include a rounded edge that is curved relative to the elongated body, may include more than one curvatures, and can be cut so the edge is smooth instead of angled. The curvature of the cut can allow for passage of the wiring 115 around the curved cut. The cutouts 160a, 160b, can be on the distal end of the outer shaft 108. The cutouts 160a, 160b, can permit clearance of the wiring 115 running from an outside surface of the jaw 206 into the inner shaft 110. The cutouts 160a, 160b, can be angled cuts to allow for this clearance. The distal end of the outer shaft 108 can additionally include one or more holes 162a, 162b, for allowing attachment of the outer shaft 108 to the forceps 100.

In FIGS. 6A-6B, an additional geometric cut on the distal end of the outer shaft 108 can be made on either side to align with the wire holders 154 of the distal wire guide 150. Here, the example geometric cut can be a stepped cut to allow clearance of the wiring 115 as it extends into the inner shaft 110. A stepped cut can include more than one angled cut in the distal end of the elongated body to allow wiring 115 to pass through. Other types and sizes of cuts can be used depending on the wiring 115 itself.

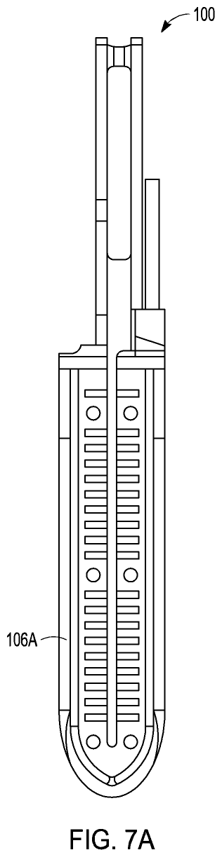

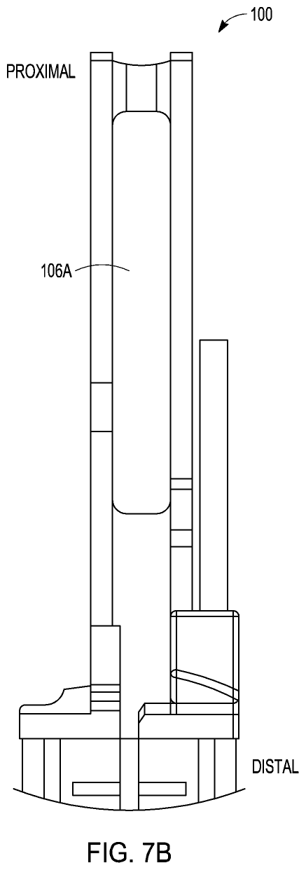

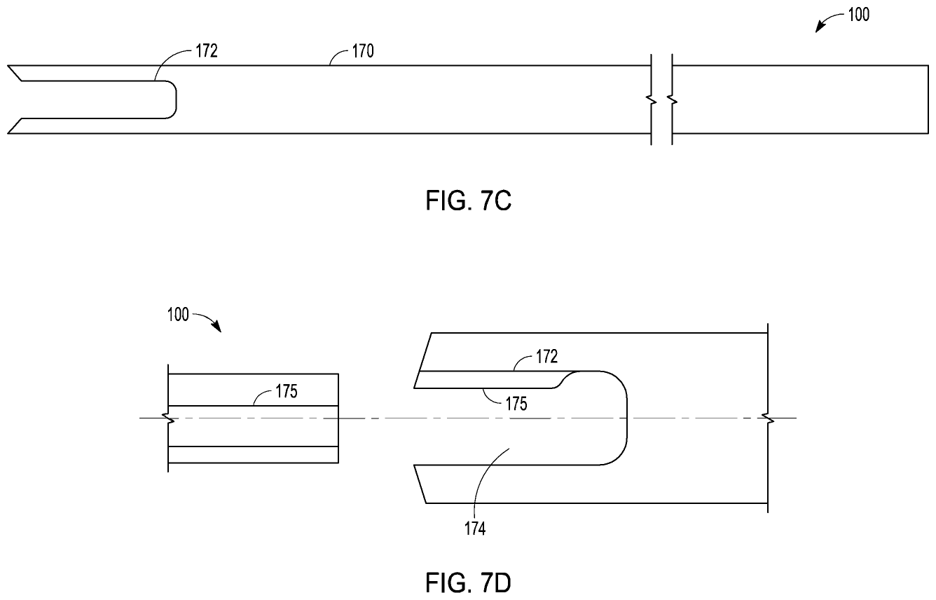

In some cases, a single action forceps can be used. FIGS. 7A-7D illustrate schematic drawings of a single action surgical forceps 100 with a welded jaw 106a. The forceps 100 can include a stationary first jaw 106a, a moveable second jaw 106b, jaw flanges 120a, 120b, 122a, 122b, a main shaft 170, a clevis juncture 172, and re-centering feature 175.

In a single action forceps, one of the jaws 106a can be welded to the main shaft, while the other jaw 106b can be actuatable relative the first jaw 106a. In this case, the first jaw 106a can be welded to the main shaft 170 of the forceps 100 at a clevis juncture 172. In some cases, the clevis juncture 172 can include a landing that is slightly offset towards the central axis of the jaw 106. For this reason, a re-centering feature 175 can be used. The re-centering feature 175 can, for example be an offset of the first jaw 106a towards a lateral side of the forceps 100. In some cases, the re-centering feature 175 can be a thickening of the first jaw 106 compared to the second jaw 106b.

With a single action forceps, the welded first jaw 106a can include two jaw flanges 120a and 120b, and the moveable second jaw 106b can include two jaw flanges 122a, 122b. The jaw flanges can be staggered. In some cases, the body of the welded jaw centered on the longitudinal axis of the shaft. Thus, the alignment of the staggered jaw flanges can be moved laterally off-center by the welding of the welded jaw 106a, such as by a few thousandths of an inch. Shown in FIGS. 7A-7D are examples of methods used to laterally re-center the jaw flanges 120, 122, within a single action forceps 100. These methods 175 can include making the first jaw 106a offset, or making the first jaw 106a thicker.

In FIGS. 7A-7D, the clevis juncture 172 into which the welded first jaw 106a is welded, can be offset to one side to adjust the alignment. The asymmetry of the clevis juncture 172 can allow for the weld gap 174 to be consumed on one side, and preserve clearance on the other side, while the jaw 106 can be aligned with the central axis of the main shaft 170.

The central line of the clevis juncture 172 can remain laterally central. However, one of the jaw flanges can be thicker than the other jaw flanges to accommodate for the difference induced by the welding and allow for the weld gap to be consumed on one side, while preserving clearance on the other side. For example, the jaw flanges 120, 122 on the welded first jaw 106a can be thicker than the moveable jaw 106b. In some cases, a single thicker jaw flange on the welded side can be used. In these configurations, for example, the jaw flanges can be appropriately laterally aligned. The features shown and discussed with reference to FIGS. 7A-7D can allow for re-centering of the device, to offset the welding of the first jaw 106a.

### VARIOUS NOTES & EXAMPLES

Each of these non-limiting examples can stand on its own, or can be combined in various permutations or combinations with one or more of the other examples.

Example 1 can include a forceps system comprising: a forceps, including a first jaw and an opposing second jaw, at least one of the first and second jaw actuatable for closing the forceps; an elongated body with a distal end and a proximal end, wherein the forceps located at the distal end of the elongated body; a wire guide at the distal end of the elongated body; and a wire extending along a lateral outside surface of at least one of the first and second jaws, through the wire guide, inside the elongated body towards the proximal end, wherein the wire guide includes a spacer separating the wire with respect to a central longitudinal axis defined by the elongated body.

Example 2 can include Example 1, where each of the first and second jaws further comprises a jaw flange extending proximally into the body from a pivot point, and wherein the wire extends around the jaw flanges within the body.

Example 3 can include any of Examples 1-2, the elongated body comprising an inner shaft situated at least partially within an outer shaft, the inner shaft and outer shaft extending between the proximal and distal ends, wherein the wire extends from the wire guide into the inner shaft.

Example 4 can include any of Examples 1-3, wherein the inner shaft comprises a drive shaft for actuating at least one of the first and second jaws.

Example 5 can include any of Examples 1-4, wherein the wire guide comprises a slot arranged for passthrough of the inner shaft from the proximal end of the body towards the forceps.

Example 6 can include any of Examples 1-5, wherein the wire guide comprises one or more wire holders for securing the wire through the wire guide.

Example 7 can include any of Examples 1-6, further comprising a handle extending from the proximal end of the elongated body, the handle shaped for operator grip of the forceps, wherein the wire internally extend towards the handle.

Example 8 can include any of Examples 1-7, further comprising one or more cutouts on the distal end of the outer shaft to permit clearance from the wiring extends from an outside surface to an inside surface.

Example 9 can include any of Examples 1-8, wherein the one or more angled cuts comprises a stepped cut.

Example 10 can include any of Examples 1-9, wherein the one or more angled cuts comprises a curved cut.

Example 11 can include any of Examples 1-10, wherein the spacer comprises one or more attachment mechanisms for securing the distal wire guide in the elongated body.

Example 12 can include any of Examples 1-11, wherein the spacer comprises a curved perimeter to allow clearance between the outer shaft and the distal wire guide.

Example 13 can include a forceps device comprising: a forceps including first jaw and an opposing second jaw, wherein the first jaw is welded to a frame and the second jaw is actuatable for opening and closing the forceps; an elongated body extending between a distal portion and a proximal portion, wherein the forceps extend from the distal portion, the body comprising the frame; and a re-centering feature in the device to offset the location of the weld and center the opening of the jaw in the device.

Example 14 can include Example 13, wherein the re-centering feature comprises an offset of the first jaw towards a lateral side of the device.

Example 15 can include any of Examples 13-14, wherein the re-centering feature comprises a thicker first jaw compared to the second jaw.

Example 16 can include a method of electrically connecting a forceps device, comprising: connecting one or more wires to an end effector on a jaw of the forceps; running the one or more wires proximally along an outside surface of the jaw; guiding the one or more wires from the outside surface of the jaw to a distal wire guide comprising a spacer and threading the one or more wires through the distal wire guide; running the one or more wires from the distal wire guide proximally to an inside surface of an elongated body of the forceps; and connecting the one or more wires to a power source.

Example 17 can include Example 16, wherein connecting one or more wires to the end effector comprises connecting one or more wires to at least one electrode on the jaw.

Example 18 can include any of Examples 16-17, wherein running the one or more wires from the distal wire guide proximally to the inside surface of the elongated body comprises running the one or more wires into a lumen of an inner shaft of the elongated body.

Example 19 can include any of Examples 16-18, further comprising running the one or more wires through a hand piece of the forceps.

Example 20 can include any of Examples 16-19, wherein connecting the one or more wires to a power source comprises connecting the handpiece to an electrical generator.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A forceps system comprising:
a forceps, including a first jaw and an opposing second jaw, at least one of the first and second jaw actuatable for closing the forceps;
an elongated body with a distal end and a proximal end, wherein the forceps located at the distal end of the elongated body;
a wire guide at the distal end of the elongated body; and
a wire extending along a lateral outside surface of at least one of the first and second jaws, through the wire guide, inside the elongated body towards the proximal end, wherein the wire guide includes a spacer separating the wire with respect to a central longitudinal axis defined by the elongated body.

2. The system of claim 1, where each of the first and second jaws further comprises a jaw flange extending proximally into the body from a pivot point, and wherein the wire extends around the jaw flanges within the body.

3. The system of claim 1, the elongated body comprising an inner shaft situated at least partially within an outer shaft, the inner shaft and outer shaft extending between the proximal and distal ends, wherein the wire extends from the wire guide into the inner shaft.

4. The system of claim 3, wherein the inner shaft comprises a drive shaft for actuating at least one of the first and second jaws.

5. The system of claim 4, wherein the wire guide comprises a slot arranged for passthrough of the inner shaft from the proximal end of the body towards the forceps.

6. The system of claim 1, wherein the wire guide comprises one or more wire holders for securing the wire through the wire guide.

7. The system of claim 1, further comprising a handle extending from the proximal end of the elongated body, the handle shaped for operator grip of the forceps, wherein the wire internally extend towards the handle.

8. The system of claim 1, further comprising one or more cutouts on the distal end of the outer shaft to permit clearance from the wiring extends from an outside surface to an inside surface.

9. The system of claim 8, wherein the one or more angled cuts comprises a stepped cut.

10. The system of claim 8, wherein the one or more angled cuts comprises a curved cut.

11. The system of claim 1, wherein the spacer comprises one or more attachment mechanisms for securing the distal wire guide in the elongated body.

12. The system of claim 1, wherein the spacer comprises a curved perimeter to allow clearance between the outer shaft and the distal wire guide.

13. A forceps device comprising:
a forceps including first jaw and an opposing second jaw, wherein the first jaw is welded to a frame and the second jaw is actuatable for opening and closing the forceps;
an elongated body extending between a distal portion and a proximal portion, wherein the forceps extend from the distal portion, the body comprising the frame; and
a re-centering feature in the device to offset the location of the weld and center the opening of the jaw in the device.

14. The forceps device of claim 13, wherein the re-centering feature comprises an offset of the first jaw towards a lateral side of the device.

15. The forceps device of claim 13, wherein the re-centering feature comprises a thicker first jaw compared to the second jaw.

16. A method of electrically connecting a forceps device, comprising:
connecting one or more wires to an end effector on a jaw of the forceps;
running the one or more wires proximally along an outside surface of the jaw;
guiding the one or more wires from the outside surface of the jaw to a distal wire guide comprising a spacer and threading the one or more wires through the distal wire guide;
running the one or more wires from the distal wire guide proximally to an inside surface of an elongated body of the forceps; and
connecting the one or more wires to a power source.

17. The method of claim 16, wherein connecting one or more wires to the end effector comprises connecting one or more wires to at least one electrode on the jaw.

18. The method of claim 16, wherein running the one or more wires from the distal wire guide proximally to the inside surface of the elongated body comprises running the one or more wires into a lumen of an inner shaft of the elongated body.

19. The method of claim 16, further comprising running the one or more wires through a hand piece of the forceps.

20. The method of claim 19, wherein connecting the one or more wires to a power source comprises connecting the handpiece to an electrical generator.
